(19) United States
(12) Patent Application Publication
(10) Pub. No.: US 2020/0132673 A1
(43) Pub. Date: Apr. 30, 2020

(54) PROTEOGLYCAN IRREGULARITIES IN ABNORMAL FIBROBLASTS AND THERAPIES BASED THEREFROM

(71) Applicant: The Regents of the University of California, Oakland, CA (US)

(72) Inventors: Nunzio Bottini, La Jolla, CA (US); Eugenio Santelli, La Jolla, CA (US); Christian Secchi, La Jolla, CA (US)

(21) Appl. No.: 16/496,933

(22) PCT Filed: Mar. 24, 2018
(86) PCT No.: PCT/US18/24220

§ 371 Date: Sep. 23, 2019

Related U.S. Application Data
(60) Provisional application No. 62/476,156, filed on Mar. 24, 2017.

(51) Int. Cl. G01N 33/50; A61P 25/28; C07K 16/18; C12N 9/48; C12Q 1/42; C12N 5/077

(52) U.S. Cl. CPC G01N 33/5047; A61P 25/28; C07K 16/18; C12N 9/48; C12Q 1/42; G01N 2800/102; G01N 2500/04; G01N 2800/52; C12Y 301/03048; G01N 2333/916; C12N 5/0652

(57) ABSTRACT

Provided herein are methods to identify agents or compounds that specifically modulate the oligomerization and/or functional activities of receptor protein tyrosine phosphatase sigma (RPTPσ) in an abnormal fibroblast cell and therapies based therefrom.

### CROSS REFERENCE TO RELATED APPLICATIONS

This application claims priority to U.S. Provisional Application Ser. No. 62/476,156, filed Mar. 24, 2017 the disclosures of which are incorporated herein by reference.

### STATEMENT REGARDING FEDERALLY SPONSORED RESEARCH

This invention was made with Government support under Grant No. R01AR066053, awarded by the National Institutes of Health. The Government has certain rights in the invention.

### TECHNICAL FIELD

Provided herein are methods to identify agents or compounds that specifically modulate the oligomerization and/or functional activities of receptor-type protein tyrosine phosphatase sigma (RPTPσ) in an abnormal fibroblast cell and therapies based therefrom as well as diagnostics to predict patient responsiveness to therapies based on such agents.

### BACKGROUND

Rheumatoid arthritis (RA) is the most common form of autoimmune arthritis, affecting more than 1.3 million Americans. Of these, about 75 percent are women. In fact, 1-3 percent of women may get rheumatoid arthritis in their lifetime. The disease most often begins between the fourth and sixth decades of life. However, RA can start at any age. RA is a chronic (long-term) disease that causes pain, stiffness, swelling and limited motion and function of many joints. While RA can affect any joint, the small joints in the hands and feet tend to be involved most often. Inflammation sometimes can affect organs as well, for instance, the eyes or lungs. Fibroblast-like synoviocytes (FLS) are specialized synovial lining cells that secrete synovial fluid and extracellular matrix (ECM) and provide structure to the joint. In RA, FLS mediate joint destruction by invading cartilage and promoting inflammation and bone erosion (see FIG. 1).

### SUMMARY

The disclosure provides a method of treating arthritis in a subject, the method comprising obtaining a biological sample comprising synovial cells, synovial-like cells and/or synovial fluid from the joint of the subject; contacting the biological sample with a test agent that inhibits clustering and/or promotes PTP activity of receptor protein tyrosine phosphatase sigma (RPTPσ); and determining whether (i) there is a change in the clustering and/or biological activity of RPTPσ, or (ii) whether the agent binds to an RPTPσ ectodomain or RPTPσ ectodomain ligand; wherein if there is an inhibition of RPTPσ clustering and/or increase in PTP activity, or binding of the test agent to the RPTPσ ectodomain or RPTPσ ectodomain ligand the subject is treated with an agent that inhibits RPTPσ clustering or promotes RPTPσ biological activity. In one embodiment, the agent is a soluble extracellular domain of RPTPσ. In another embodiment, the agent is an RPTPσ Ig1&2 polypeptide. In yet another embodiment, the agent is an antibody that specifically interacts with the RPTPσ ectodomain and inhibits clustering. In still another or further embodiment, the method further comprises measuring the level of syndecan-4.

The disclosure also provides a method of screening a subject having or at risk of having arthritis, the method comprising obtaining fibroblast-like synoviocytes (FLS) from a subject; contacting the FLS cells with an agent that inhibits clustering and/or promotes PTP activity of receptor protein tyrosine phosphatase sigma (RPTPσ); and determining whether (i) there is a change in the clustering and/or biological activity of RPTPσ on the FLS cells, or (ii) whether the agent binds to an RPTPσ ectodomain ligand on the FLS cells, wherein if there is an inhibition of RPTPσ clustering and/or increase in PTP activity, or binding of the test agent to an RPTPσ ectodomain ligand is indicative of rheumatoid arthritis. In one embodiment, the agent is a soluble extracellular domain of RPTPσ. In yet another embodiment, the agent is an RPTPσ Ig1&2 polypeptide. In still another embodiment, the agent is an antibody that specifically interacts with the RPTPσ ectodomain and inhibits clustering. In another or further embodiment, the method comprises measuring the level of syndecan-4.

The disclosure also provides a method of screening an agent that modulates receptor protein tyrosine phosphatase sigma (RPTPσ) clustering and/or activity, comprising contacting fibroblast-like synoviocytes from a rheumatoid arthritis subject with a test agent; determining (i) a change in the clustering and/or biological activity of RPTPσ in the presence and absence of the test agent, or (ii) whether the agent binds to the RPTPσ ectodomain or one of its ligands, wherein inhibition of clustering or a declustering of RPTPσ is indicative of an agent that modulates RPTPσ.

The disclosure also provides a method to determine whether a compound or an agent modulates the clustering and/or functional activity of receptor protein tyrosine phosphatase sigma (RPTPσ), comprising contacting a RPTPσ of an abnormal fibroblast cell or suspected abnormal fibroblast cell, and a normal or osteroarthritis (OA) fibroblast cell with the compound or the agent; and determining whether the compound or the agent modulates the clustering and/or functional activity of the RPTPσ of the abnormal fibroblast or suspected abnormal fibroblast cell but does not modulate the clustering and/or functional activity of a RPTPσ from a normal or OA fibroblast cell. In one embodiment, the method measures whether the compound or the agent promotes the clustering and/or inhibits PTP functional activity of the RPTPσ of the abnormal or suspected abnormal fibroblast cell. In a another or further embodiment, the method measures whether the compound or the agent inhibits the clustering and/or promotes PTP functional activity of the RPTPσ of the abnormal or suspected abnormal fibroblast cell. In yet another embodiment, the fibroblast cell is a fibroblast-like synoviocyte. In still another or further embodiment of the foregoing, the abnormal fibroblast or suspected abnormal fibroblast is a fibroblast-like synoviocyte from a subject with rheumatoid arthritis. In another or further embodiment of the foregoing, the abnormal fibroblast or suspected abnormal fibroblast is a fibroblast from a subject with idiopathic pulmonary fibrosis, Dupuytren's disease, scleroderma or cancer.

The disclosure also provides a pharmaceutical composition comprising a compound or agent determined from the method of the disclosure that modulates clustering or activity of receptor protein tyrosine phosphatase sigma (RPTPσ), and a pharmaceutical carrier.

The disclosure also provides a method of determining a therapeutic treatment or prognosis of a subject receiving treatment for arthritis, idiopathic pulmonary fibrosis, Dupuytren's disease, scleroderma or cancer, the method comprising obtaining a sample from the subject comprising fibroblast or fibroblast-like cells; contacting the cells with the therapeutic agent; measuring the clustering and/or functional activity of receptor protein tyrosine phosphatase sigma (RPTPσ), wherein a decrease or inhibition of the clustering and/or promotion of PTP functional activity of the RPTPσ is indicative of a beneficial treatment or prognosis.

The disclosure also provides a method to treat a disease or disorder in a subject that has proteoglycan irregularities associated with receptor protein tyrosine phosphatase sigma (RPTPσ) clustering and/or function activity, comprising administering to the subject the pharmaceutical composition of that inhibits or promotes clustering of receptor protein tyrosine phosphatase sigma (RPTPσ). In one embodiment, the disease or disorder is selected from rheumatoid arthritis, idiopathic pulmonary fibrosis, Dupuytren's disease, scleroderma or cancer.

Identified are methods to predict the responsiveness of patients to agents that change proteoglycan function. Also identified herein are agents (e.g., recombinant receptor-type protein tyrosine phosphatase sigma immunoglobulin-like domains (Ig) 1&2) that modulate the biological activity of rheumatoid FLS but not osteoarthritis (OA) FLS. The difference in activity is largely attributed to the difference in the proteoglycan (PG) composition between RA FLS and OA FLS, whereby recombinant RPTPσ Ig1&2 cannot bind efficiently the PGs on OA FLS. By implication, the proteoglycan (PG) switch is already in an active state on the surface of OA FLS and cannot be flipped further by such agents.

In a particular embodiment, the disclosure provides a method to determine whether a compound or an agent modulates the clustering and/or functional activity of RPTPσ in an abnormal fibroblast, comprising: contacting a RPTPσ of an abnormal fibroblast cell or suspected abnormal fibroblast cell, and a normal or OA fibroblast with the compound or the agent; and determining whether the compound or the agent modulates the clustering and/or functional activity of RPTPσ of the abnormal fibroblast or suspected abnormal fibroblast cell but does not modulate the clustering and/or functional activity of RPTPσ from a normal or OA fibroblast cell. In a further embodiment, the method is determining whether the compound or the agent promotes the clustering and/or inhibits functional activity of RPTPσ of the abnormal or suspected abnormal fibroblast cell. In an alternate embodiment, the method is determining whether the compound or the agent inhibits the clustering and/or promotes functional activity of RPTPσ of the abnormal or suspected abnormal fibroblast cell. For example, if the clustering is inhibited then the functional activity is promoted and if the clustering is promoted then the functional activity is inhibited. In yet a further embodiment the fibroblast cell is a fibroblast-like synoviocyte. In another embodiment, the abnormal fibroblast or suspected abnormal fibroblast is a fibroblast-like synoviocyte from a subject with rheumatoid arthritis. In an alternate embodiment, the abnormal fibroblast or suspected abnormal fibroblast is a fibroblast from a subject with idiopathic pulmonary fibrosis, Dupuytren's disease, scleroderma or cancer.

In a certain embodiment, the disclosure also provides for a pharmaceutical composition which comprises a compound or agent determined from a method disclosed herein that modulates clustering and/or functional activity of RPTPσ of the abnormal fibroblast cell, and a pharmaceutical carrier.

In a particular embodiment, the disclosure further provides for a method to treat a disease or disorder in a subject that has proteoglycan irregularity associated with receptor protein tyrosine phosphatase sigma (RPTPσ) activation or inactivation, comprising: administering to the subject the pharmaceutical composition of the disclosure. In yet a further embodiment, the disease or disorder is selected from rheumatoid arthritis, idiopathic pulmonary fibrosis, Dupuytren's disease, scleroderma or cancer. In a particular embodiment, the disease or disorder is rheumatoid arthritis.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1 provides a cartoon demonstrating the roles of FLS in RA. FLS play a critical part in many pathogenic events in the RA synovium. They can contribute to pathology through a reduced ability to undergo apoptosis (forming pannus), the production of proteases that degrade the extracellular matrix, and invasion into cartilage. In addition, FLS produce a variety of molecules that modulate growth, inflammation, angiogenesis, and cell recruitment, and induce activation of and cytokine production by immune cells. Abbreviations: CCL2, CC-chemokine ligand 2; CXCL10, CXC-chemokine ligand 10; FLS, fibroblast-like synoviocytes; GM-CSF, granulocyte-macrophage colony-stimulating factor; MMP, matrix metalloproteinase; PDGF, platelet-derived growth factor; RA, rheumatoid arthritis; TGF-β, transforming growth factor β; VEGF, vascular endothelial growth factor.

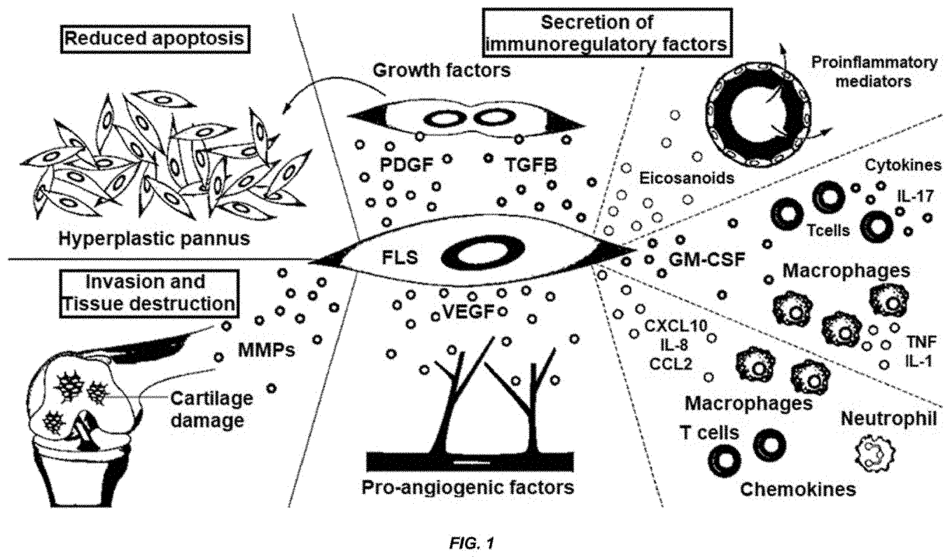

FIG. 2 demonstrates that RPTPσ Ig1&2-Fc specifically inhibits RA FLS migration in a dose response manner. RA FLS (N=3) and OA FLS (N=3) were starved for 24 hours and allowed to migrate through transwells in response to 5% FBS in the presence of RPTPσ Ig1&2 (respectively 20 nM, 10 nM, 5 nM and 2.5 nM) or vehicle/IgG1Fc as control. Mean±SEM fold change of migration relative to the vehicle-treated cells from the same experiment is shown. Data are from three independent experiments (n=72 fields; *P<0.05, Mann-Whitney).

FIG. 3A-B shows (A) that RPTPσ Ig1&2 specifically delays RA FLS healing in comparison to OA FLS. RA FLS (N=3) and OA FLS (N=3) monolayers were serum-starved before scratch wounding and stimulation with 10% FBS in the presence of 20 nM RPTPσ Ig1&2 or vehicle. Wound width was measured at three points at the indicated times. Mean±s.e.m wound width (****P<0.01, ANOVA). (B) shows Syndecan-4 is highly expressed in RA FLS. RA FLS (N=8) and OA FLS (N=9) were serum-starved for 48 h and then syndecan-4 mRNA levels were measured by qPCR. Graph shows means+/−standard error of the mean (s.e.m.) relative expression following normalization to the housekeeping gene GAPDH. Data were analyzed using the two-tailed Mann-Whitney test (*, P<0.05).

FIG. 4A-B demonstrates that RPTPσ Ig1&2 binds preferentially RA FLS. RA FLS (N=3) and OA FLS (N=3) were stained with chemically biotinylated Ig1&2 and AviTag biotinylated Ig1&2. (A) Representative Flow diagrams of the comparison between staining of biotinylated RPTPσ Ig1&2 in OA and RA FLS analyzed by FACS. All data are expressed as normalized to mode. (B) Median Fluorescence Intensity difference between biotinylated RPTPσ Ig1&2 (AviTag and chemically) stained and unstained by flow cytometry analysis in OA FLS vs RA FLS, *P<0.05, student t-test.

FIG. 5 presents a model for RPTPσ-dependent PG switch in FLS from Doody et al. (Sci Transl. Med. 7(288):288ra76, 2015). RPTPσ interacts with the HS PG syndecan-4 on the surface of FLS and is maintained in an inactive oligomeric state. Tyrosine phosphorylation of ezrin downstream of the PDGFR promotes ezrin localization to the actin cytoskeleton, enabling cell migration and invasion. Disruption of the RPTPσ-HS interaction by the HS-binding decoy RPTPσ Ig1&2 fragment displaces RPTPσ from HS. This leads to dephosphorylation of ezrin and disassociation of ezrin from the actin cytoskeleton, decreasing FLS migration, invasion, and attachment to cartilage.

FIG. 6 does not show a difference in HS sulfation between OA and RA FLS using GAG mass spectrometric analysis. RA FLS (N=3) and OA FLS (N=3) were cultured up to 3rd passage. Media containing secreted HS and media from trypsinized cells containing cell surface HS was collected 3 times during cell culture. Then GAGs were purified by DEAE sepharose beads and lyophilized until dry. Water-resuspended GAGs were then digested with 2 mU of Hep Lyase I, II, III, labeled with an aniline tag and then HS quantitative analysis was carried out by mass spectrometry as described in Lawrence R. et al. JBC, 2008. Graph shows molar percentage of each HS sulfate modifications for RA FLS vs OA FLS samples. Data were analyzed using the two-tailed Mann-Whitney test (n.s.=P>0.05).

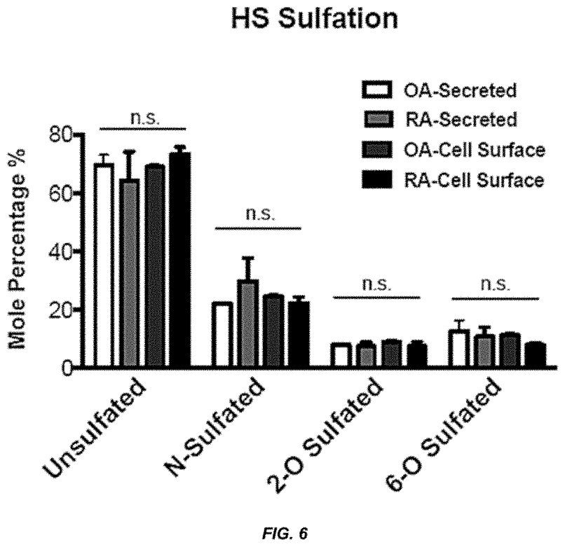

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the agent” includes reference to one or more agents, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

The term “agent” as used herein refers to any molecule or compound that can be used in the methods and compositions of the disclosure. An agent can be a biological agent such as a protein, peptide, polypeptide (e.g., an antibody or fragment thereof), nucleic acid (e.g., RNAi molecule); a macromolecule or small molecule agent.

“Biological sample” or “sample” refer to materials obtained from or derived from a subject or patient. A biological sample includes sections of tissues such as biopsy and autopsy samples, and frozen sections taken for histological purposes. Such samples include bodily fluids. Thus, a biological sample includes, for example, synovial fluid, blood and blood fractions or products (e.g., serum, plasma, platelets, red blood cells, and the like), media from cultured cells (e.g., primary cultures, explants, and transformed cells) stool, urine, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the disclosure. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), among other factors. Representative biopsy techniques include excisional biopsy, incisional biopsy, needle biopsy, surgical biopsy, and bone marrow biopsy.

As used herein “clustering” of RPTPσ refers to the association of multiple monomers of RPTPσ or oligomerization of RPTPσ. The clustering of RPTPσ causes “deactivation” of PTP activity (i.e., the ability to dephosphorylate proteins is decreased or deactivated). In contrast, “declustering” promotes PTP activity and the dephosphorylating of proteins. Syndecan-4, for example, promotes clustering of RPTPσ and thus decreases/inhibits PTP activity.

The term “diagnosis” refers to a relative probability that a disease (e.g. an autoimmune, inflammatory autoimmune, cancer, infectious, immune, or other disease) is present in the subject. The disclosure provides methods of diagnosing a disease or disorder associated with aberrant clustering and/or functional activity of RPTPσ. For example, diagnosing a condition can be made by determining the amount of clustering and/or activity of RPTPσ compared to a normal control population or from tissue or cells of osteoarthritis (e.g., OA FLS), subject or measurement, wherein a statistically significant difference in clustering and/or function activity of RPTPσ is indicative/diagnostic of a disease or disorder. The disclosure also provides a method of diagnosing a subject by measuring the amount of syndecan-4 and comparing the amount of syndecan-4 to a normal control, wherein if syndecan-4 levels are higher than a normal control then the subject has or is at risk of having an autoimmune, inflammatory, cancer, or infection. For example, the disease or disorder can be rheumatoid arthritis.

The terms “dose” and “dosage” are used interchangeably herein. A dose refers to the amount of active ingredient given to an individual at each administration, or to an amount administered in vitro or ex vivo. For the methods and compositions provided herein, the dose may generally depend on the required treatment for the disease (e.g. an autoimmune, inflammatory autoimmune, or other disease), and the biological activity of a compound or agent disclosed herein. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “effective amount,” “therapeutically effective amount,” “therapeutically effective dose or amount” and the like as used herein is meant an amount (e.g., a dose) that produces effects for which it is administered (e.g., inhibiting or promoting clustering and/or functional activity of RPTPσ). An effective dose can be characterized in cell culture to modulate a particular biological readout (e.g., expression or a gene or protein, clustering, etc.). The exact dose and formulation will depend on the purpose of the research or treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “pharmaceutically acceptable” is used synonymously with “physiologically acceptable” and “pharmacologically acceptable”. A pharmaceutical composition will generally include agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and/or absorption by a subject and can be included in the compositions disclosed herein without causing a significant adverse toxicological effect on the patient. Unless indicated to the contrary, the terms “active agent,” “active ingredient,” “therapeutically active agent,” “therapeutic agent” and like are used synonymously. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, polyethylene glycol, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein. One of skill in the art will recognize that other pharmaceutical excipients are useful in the methods and compositions disclosed herein.

The term “prognosis” refers to a relative probability that a certain future outcome may occur in the subject with respect to a disease state. For example, in the present context, prognosis can refer to the likelihood that an individual will develop a disease (e.g. an autoimmune, inflammatory autoimmune, cancer, infectious, immune, or other disease), or the likely severity of the disease (e.g., extent of abnormal effect and duration of disease), or a likelihood of progression or regression of a disease. The terms are not intended to be absolute, as will be appreciated by any one of skill in the field of medical diagnostics.

“PTPRS” or “RPTPσ” refers to protein tyrosine phosphatase receptor type S (or sigma), which is a member of the protein tyrosine phosphatase (PTP) family. The amino acid sequence of RPTPσ can be found, for example, at UniProtKB/Swiss-Prot Accession No. Q13332 (human) and BOV2N1 (Mus musculus), and Q64605 (Rattus norvegicus) (the contents of which are incorporated herein by reference). The nucleic acid sequence of RPTPσ can be found, for example, at GenBank Accession No. NC 000019.9 (the content of which is incorporated herein by reference). RPTPσ includes an intracellular domain, a transmembrane domain, and an extracellular domain. The term transmembrane domain refers to the portion of a protein or polypeptide that is embedded in and, optionally, spans a membrane. The term intracellular domain refers to the portion of a protein or polypeptide that extends into the cytoplasm of a cell. The term extracellular domain refers to the portion of a protein or polypeptide that extends into the extracellular environment. The extracellular domain of RPTPσ includes immunoglobulin-like domain 1 (Ig1), immunoglobulin-like domain 2 (Ig2) and immunoglobulin-like domain 2 (Ig3). The amino acid sequence of Ig1, Ig2 and Ig3 are known.

An “RPTPσ compound” or “RPTPσ agent” as used herein refer to a compound or agent that binds to RPTPσ or to ligands that normally bind to RPTPσ so as to modulate (e.g., inhibiting or promoting) clustering and/or functional activity of RPTPσ. A RPTPσ compound or RPTPσ agent preferentially binds to RPTPσ as compared to other macromolecular biomolecules present in an organism or cell, for example, when the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In a particular embodiment, the RPTPσ compound or RPTPσ agent preferentially binds to RPTPσ so as to modulate clustering and/or functional activity of RPTPσ. In an alternate embodiment, the RPTPσ compound or RPTPσ agent preferentially binds to a ligand which normally binds to RPTPσ so as to modulate clustering and/or functional activity of RPTPσ.

In certain embodiments herein, an “RPTPσ compound” or “RPTPσ agent” is a small chemical molecule RPTPσ ligand mimetic. The term “small chemical molecule” and the like, as used herein, refers to a molecule that has a molecular weight of less than two thousand (2000) Daltons, less than one thousand (1000) Daltons, less than five hundred (500) Daltons, less than one hundred (100) Daltons, or range between or including any two of the foregoing values. In one or more embodiments disclosed herein, the RPTPσ ligand mimetic is recombinant RPTPσ Ig1&2. In another embodiment, the “RPTPσ compound” or “RPTPσ agent” is a peptide, polypeptide or protein. In another embodiment, the “RPTPσ compound” or “RPTPσ agent” is a macromolecule.

The terms “subject,” “patient,” “individual,” etc. are not intended to be limiting and can be generally interchanged. That is, an individual described as a “patient” does not necessarily have a given disease, but may be merely seeking medical advice. Moreover, a subject, patient or individual can be any mammal including primates, canines, felines, bovines, equines, porcine, etc. Preferably the subject is a human subject.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

Fibroblast-like synoviocytes (FLSs) are local joint lining cells which mediate cartilage destruction and promote inflammation and bone erosion in rheumatoid arthritis (RA). The behavior of an FLS is regulated by several intracellular pathways involving protein tyrosine phosphorylation. A transmembrane PTP belonging to the R2A subclass, called RPTPσ (gene RPTPσ), is a highly-expressed phosphatase in arthritic FLS. In neurons, engagement of RPTPσ N-terminal extracellular immunoglobulin-like domains 1 and 2 (Ig1&2) by heparan sulfate (HS) or chondroitin sulfate (CS) glycosaminoglycan (GAG) moieties of various proteoglycans (PGs) controls axonal extension. The interaction of RPTPσ Ig1&2 with HS-containing PG induces RPTPσ clustering and/or oligomerization and functional inactivation of RPTPσ, which promotes axonal extension. On the other hand, CS-containing PG can compete with HS-containing PG for binding to the same RPTPσ Ig1&2 domains, de-clustering RPTPσ and inhibiting axonal extension. This mechanism has been termed the “PG switch”, and it mediates inhibition of axonal growth through CS-rich repair tissue after spinal cord injuries. For example an “active” PG switch comprises a declustered RPTPG and an inactive PG switch comprises a clustered RPTPσ.

The joint is composed of highly PG-rich tissue, and CS is the predominant GAG in cartilage. On the other hand, HS-containing PGs are primarily located on cell surfaces where they mediate interaction between cells and surrounding ECM. In FLS, the HS PG syndecan-4 is required for the attachment of FLS to cartilage, an important pathogenic FLS behavior.

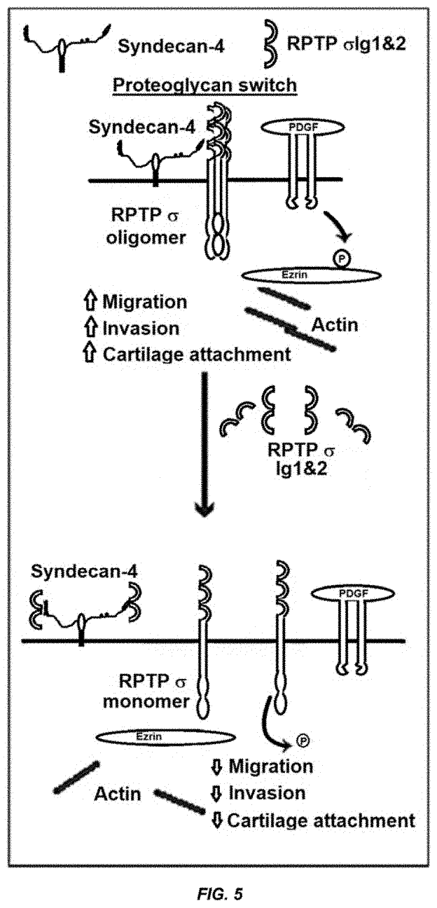

In FLS, RPTPσ expression is upregulated and during mouse arthritis progression clustering is promoted via interactions of syndecan-4 with RPTPσ, thus decreasing/inhibiting PTP activity. Further, the HS PG syndecan-4 is required to keep RPTPσ inactive thus promoting FLS invasiveness and attachment to cartilage which are important pathogenic FLS behaviors (see FIG. 5). The manipulation of the PG switch using a recombinant PG binding decoy RPTPσ Ig1&2 protein inhibited FLS invasion, migration, and attachment to cartilage in vitro and in vivo and ameliorated arthritis in mice by activating the PG switch.

RA FLS have intrinsic abnormalities when compared to FLS from healthy people, which sustain inflammation and promote cartilage and bone destruction in RA. The glycobiology or glycopathology of RA FLS remains unknown. During mouse arthritis progression, it has been shown that the transmembrane phosphatase RPTPσ is upregulated in FLS. On the surface of FLS, RPTPσ is kept in an inactive state by the interaction of its extracellular domain with Heparan Sulfate Proteoglycans and can be activated by the displacement of such interaction. This mechanism, named the PG switch, can be leveraged by a recombinant PG binding decoy protein called RPTPσ Ig1&2 which was shown to inhibit RA FLS migration and invasion in vitro and in vivo and to improve the course of arthritis after injection in mice.

This disclosure describes the surprising finding that Ig1&2 treatment only acts on abnormal, but not OA or normal FLS. In particular, FLS glycopathology gives rise to PG abnormalities which underlie the differential action of Ig1&2 on RA vs OA FLS. Accordingly, by exploiting the differences in PG composition in RA FLS and normal FLS, a therapy can be tailored to specifically treat RA. Thus, RPTPσ Ig1&2 cannot bind the PGs on OA FLS, which would imply that the PG switch is already in an active state on the surface of OA FLS and cannot be flipped further by recombinant Ig1&2. Moreover, known these differences one could target additional protein/protein systems (e.g., the PG switch) by using active drug products in view of the PG differences between RA vs OA FLS.

In one embodiment, the disclosure provides a method to identify agents that modulate the clustering and/or functional activity of RPTPσ, comprising contacting an abnormal fibroblast having proteoglycan abnormalities with a test agent and measuring the clustering and/or functional activity of RPTPσ and/or RPTPσ dependent cellular behavior before and after contacting with the test agent, wherein a change in clustering and/or functional activity of RPTPσ and/or RPTPσ dependent cellular behavior is indicative of an agent the modulates RPTPσ activity.

In another embodiment, the disclosure provides a method of identifying a therapy for a subject suffering from arthritis. The method comprises obtaining a biological sample from the subject and measure(i) expression of syndecan-4 in the biopsy or in the synovial fibroblasts or in the synovial fluid, (ii) measuring the ability to inhibit the clustering (or oligomerization) of RPTPσ or (iii) the ability to promote RPTPσ activity in a cell of the biopsy. In one embodiment, the method includes obtaining FLS cells from the subject, culturing the FLS cells in the presence and absence of Ig1&2 and determining whether the clustering and/or functional activity of RPTPσ is changed. If the clustering and/or functional activity of RPTPσ is changed then the subject has an RA type arthritis and/or can be treated with an inhibitor of RPTPσ clustering. In another embodiment, immuno-histochemistry or immunofluorescence is used to identify RPTPσ (e.g., clustering/declustering) or assays are performed to measure activity in response to modulators of RPTPσ clustering (e.g., measuring phosphorylation/dephosphorylation). In an alternative or further embodiment, the synovial fluid of the subject is obtained and the level of syndecan-4 is measured. If the level of syndecan-4 in the synovial fluid is greater by at least 5%, 10%, 15%, 20%, 25%, 40%, 50%, 60%, 75%, 80%, 90%, or at least 100% greater; or 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater, then the subject has an RA type arthritis. The disclosure further provides that if a subject has increased levels of syndecan-4 and/or shows clustering of RPTPσ and/or shows dephosphorylation in response to an agent the “declusters” RPTPσ, then the subject can be treated with an agent that inhibits RPTPσ clustering (e.g., Ig1&2 domains).

The disclosure also provides methods of identifying a candidate RPTPσ de-clustering agent useful for treating RA or RPTPσ clustering diseases and disorders, the method comprising contacting a test agent with an RA-FLS cell and detecting de-clustering of the RPTPσ peptides, thereby identifying a candidate RPTPσ de-clustering agent. Optionally, the method of identifying a candidate RPTPσ de-clustering agent includes contacting a test agent with a RA-FLS culture and heparan sulfate and determining whether the test agent inhibits or reduces binding of the RPTPσ to heparan sulfate, inhibition or reduction of binding indicating the test agent is a RPTPσ de-clustering agent. As mentioned above an “agent” can be a nucleic acid, peptide, antibody, macromolecule, or small molecule. Various methods are available to assess whether an agent is effective at declustering RPTPσ. For example, a cell-based readout assay for RPTPσ function can be used. Declustering of RPTPσ can be observed by detecting a decrease in tyrosine phosphorylation of RPTPσ substrates or downstream signaling intermediates. Declustering can also be observed by detecting a decrease in migration or invasion of FLS cells. Other assays include, but are not limited to, FRET-based assays, and gel-filtration. For example, in such assays, cells can be treated with HS to induce RPTPσ clustering in cells that have low clustering (e.g., OA FLS cells), and then treated with the test agents to test for declustering of RPTPσ. Similarly, inhibition binding of RPTPσ to heparan sulfate can be detected using any appropriate method known in the art. For example, an agent can be identified as an agent that inhibits or reduces binding of RPTPσ to heparan sulfate by performing an assay in which binding of RPTPσ to heparan sulfate can be detected (e.g., an immunoassay). The agent inhibits or reduces binding of RPTPσ to heparan sulfate if binding of RPTPσ to heparan sulfate can be detected in the absence of the agent but is no longer detected or binding is reduced in the presence of the agent. Optionally, binding can be detected by determining whether the agent to be tested competitively inhibits heparan sulfate from binding to RPTPσ. Optionally, an agent can be identified as an agent that inhibits or reduces binding of RPTPσ to heparan sulfate by performing an assay that measures the function or activity of RPTPσ.

By way of example, a test agent can be identified as a RPTPσ de-clustering agent in animal models (e.g., animal models comprising human RA FLS cells or subjects) by determining if the agent reduces the severity of one or more symptoms of the autoimmune or inflammatory disease or condition. Thus, by way of example, in the provided screening methods, the contacting step comprises administering the agent to a subject with an autoimmune or inflammatory disease and the determining step comprises determining whether the agent prevents or reduces one or more symptoms of the disease in a subject. In one embodiment, the disease is rheumatoid arthritis. Such screening methods can be carried out using, for example, animal models comprising human RA FLS cells of inflammatory and autoimmune disease.

The methods described herein include methods (also referred to herein as “screening assays”) for identifying compounds/agents that modulate (i.e., increase or decrease) clustering and/or functional activity of RPTPσ. Such compounds include, e.g., polypeptides, peptides, antibodies, peptidomimetics, peptoids, small inorganic molecules, small non-nucleic acid organic molecules, nucleic acids (e.g., anti-sense nucleic acids, siRNA, oligonucleotides, synthetic oligonucleotides), carbohydrates, or other agents that bind to a RPTPσ (e.g., syndecan-4) and/or have a stimulatory or inhibitory effect on, for example, clustering and/or functional activity of RPTPσ. Compounds thus identified can be used to modulate the expression or activity of target genes or RPTPσ in a therapeutic protocol.

In general, screening assays involve assaying the effect of a test agent on expression or activity of a target nucleic acid or RPTPσ in a test sample (i.e., a sample containing the target nucleic acid or RPTPσ). Expression or activity in the presence of the test compound or agent can be compared to expression or activity in a control sample (i.e., a sample containing the RPTPσ that is incubated under the same conditions, but without the test compound). A change in the expression or activity of the target nucleic acid or RPTPσ in the test sample compared to the control indicates that the test agent or compound modulates expression or activity of the target nucleic acid or RPTPσ and is a candidate agent.

Compounds can be tested for their ability to modulate one or more activities mediated by a RPTPσ described herein. For example, compounds can be tested for their ability to modulate clustering and/or functional activity of RPTPσ. Methods for screening such compounds can be in vivo (e.g., in animal models) or in vitro (e.g., in cell culture). In one embodiment, the method comprises contacting an animal model of RA with the test compound and determining any change in joint inflammation or other symptoms of the animal. In one embodiment, the method includes contacting RA-FLS cells with a test agent/compound and determining whether there is a change in clustering and/or functional activity of RPTPσ and/or phosphorylation or downstream second messengers in the cells.

The test compounds/agents used in the methods can be obtained using any of the numerous approaches in the art including combinatorial library methods, including: biological libraries; peptoid libraries (libraries of molecules having the functionalities of peptides, but with a novel, non-peptide backbone which are resistant to enzymatic degradation but which nevertheless remain bioactive; e.g., Zuckermann et al. (1994) J. Med. Chem. 37:2678); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the literature, for example in: DeWitt et al., Proc. Natl. Acad. Sci. USA, 90:6909, 1993; Erb et al., Proc. Natl. Acad. Sci. USA, 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl., 33:2061, 1994; and Gallop et al., J. Med. Chem., 37:1233, 1994.

Libraries of compounds may be presented in solution (e.g., Houghten, Bio/Techniques, 13:412421,1992), or on beads (Lam, Nature, 354:82-84, 1991), chips (Fodor, Nature 364:555-556, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA, 89:1865-1869, 1992) or phage (Scott and Smith, Science, 249:386-390, 1990; Devlin, Science, 249:404-406, 1990; Cwirla et al., Proc. Natl. Acad. Sci. USA, 87:6378-6382, 1990; and Felici, J. Mol. Biol., 222:301-310, 1991).

In one embodiment, a cell-based assay is employed in which a cell that expresses RPTPσ is contacted with a test compound. The ability of the test compound to modulate clustering and/or functional activity of RPTPσ is then determined. The cell, for example, can be a FLS cell from RA or OA tissue sources of mammalian origin, e.g., human.

The ability of the test compound to bind to RPTPσ or modulate clustering and/or functional activity of RPTPσ, can also be evaluated. This can be accomplished, for example, by coupling the compound, e.g., with a radioisotope or enzymatic label such that binding of the compound, to RPTPσ can be determined by detecting the labeled compound, in a complex. Alternatively, the RPTPσ can be coupled with a radioisotope or enzymatic label to monitor the ability of a test compound to modulate clustering and/or functional activity of RPTPσ. For example, compounds can be labeled with 125I, 35S, 14C, or 3H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The interaction between two molecules (e.g., an agent and RPTPσ) can also be detected using fluorescence energy transfer (FET) (see, for example, Lakowicz et al., U.S. Pat. No. 5,631,169; Stavrianopoulos et al., U.S. Pat. No. 4,868,103). A fluorophore label on the first, “donor” molecule is selected such that its emitted fluorescent energy will be absorbed by a fluorescent label on a second, “acceptor” molecule, which in turn is able to fluoresce due to the absorbed energy. Alternately, the “donor” protein molecule may use the natural fluorescent energy of tryptophan residues. Labels are chosen that emit different wavelengths of light, such that the “acceptor” molecule label may be differentiated from that of the “donor.” Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorimeter).

This disclosure further pertains to novel agents identified by the above-described screening assays or related assays known in the art. Accordingly, it is within the scope of this disclosure to further use an agent (compound) identified as described herein (e.g., a RPTPσ modulating agent, an antisense nucleic acid molecule, an siRNA, a RPTPσ-specific antibody, an RPTPσ-ligand specific antibody (e.g., antibody to syndecan-4) or a RPTPσ-binding partner) in an appropriate animal model to determine the efficacy, toxicity, side effects, or mechanism of action, of treatment with such an agent. Furthermore, novel agents identified by the above-described screening assays (or other assays known in the art) can be used for treatments as described herein.

Isolated RPTPσ, fragments thereof, and variants thereof are provided herein. These polypeptides can be used, e.g., as immunogens to raise antibodies, in screening methods, or in methods of treating subjects, e.g., by administration of the RPTPσ's soluble domains (e.g., extracellular or Ig1&2 domains). An “isolated” or “purified” polypeptide or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of polypeptides in which the polypeptide of interest is separated from cellular components of the cells from which it is isolated or recombinantly produced. Thus, a polypeptide that is substantially free of cellular material includes preparations of polypeptides having less than about 30%, 20%, 10%, or 5% (by dry weight) of heterologous protein (also referred to herein as “contaminating protein”). In general, when the polypeptide or biologically active portion thereof is recombinantly produced, it is also substantially free of culture medium, i.e., culture medium represents less than about 20%, 10%, or 5% of the volume of the protein preparation. In general, when the polypeptide is produced by chemical synthesis, it is substantially free of chemical precursors or other chemicals, i.e., it is separated from chemical precursors or other chemicals that are involved in the synthesis of the polypeptide. Accordingly such preparations of the polypeptide have less than about 30%, 20%, 10%, or 5% (by dry weight) of chemical precursors or compounds other than the polypeptide of interest.

Expression of RPTPσ can be assayed to determine the amount of expression. Methods for assaying protein expression are known in the art and include Western blot, immunoprecipitation, and radioimmunoassay.

As used herein, a “biologically active portion” of a RPTPσ includes a fragment of a RPTPσ that participates in an interaction between a RPTPσ and a proteoglycan (e.g., syndecan-4). Biologically active portions of a RPTPσ include peptides including amino acid sequences sufficiently homologous to the amino acid sequence of a RPTPσ that includes fewer amino acids than a full-length RPTPσ, and exhibits at least one activity of a RPTPσ (e.g., binding syndecan-4). Typically, biologically active portions include a domain or motif with at least one activity of the RPTPσ. A biologically active portion of a RPTPσ can be a polypeptide that is, for example, 10, 25, 50, 100, 200 or more amino acids in length. Biologically active portions of a RPTPσ can be used as targets for developing agents that modulate a RPTPσ mediated activity, e.g., compounds that inhibit RPTPσ activity or the ability of (or compete with) the binding of RPTPσ extracellular domain with a proteoglycan or cognate.

An RPTPσ, or a fragment thereof (e.g., an extracellular domain), can be used as an immunogen to generate antibodies using standard techniques for polyclonal and monoclonal antibody preparation. The full-length polypeptide or protein can be used or, alternatively, antigenic peptide fragments can be used as immunogens. The antigenic peptide of a protein comprises at least 8 (e.g., at least 10, 15, 20, or 30) amino acid residues of the amino acid sequence of a RPTPσ, and encompasses an epitope of a RPTPσ such that an antibody raised against the peptide forms a specific immune complex with the polypeptide.

An immunogen typically is used to prepare antibodies by immunizing a suitable subject (e.g., rabbit, goat, mouse or other mammal). An appropriate immunogenic preparation can contain, for example, a recombinantly expressed or a chemically synthesized polypeptide. The preparation can further include an adjuvant, such as Freund's complete or incomplete adjuvant, or similar immunostimulatory agent.

Polyclonal antibodies can be prepared as described above by immunizing a suitable subject with a RPTPσ as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein, Nature, 256:495-497, 1975, the human B cell hybridoma technique (Kozbor et al., Immunol. Today, 4:72, 1983), the EBV-hybridoma technique (Cole et al., Monoclonal Antibodies and Cancer Therapy, Alan R. Liss, Inc., pp. 77-96, 1985) or trioma techniques. The technology for producing hybridomas is well known (see generally Current Protocols in Immunology, 30 1994, Coligan et al. (eds.) John Wiley & Sons, Inc., New York, N.Y.). Hybridoma cells producing a monoclonal antibody are detected by screening the hybridoma culture supernatants for antibodies that bind the polypeptide of interest, e.g., using a standard ELISA assay.

As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™. Phage Display Kit, Catalog No. 240612). Additionally, examples of methods and reagents particularly amenable for use in generating and screening antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology, 9:1370-1372, 1991; Hay et al., Hum. Antibod. Hybridomas, 3:81-85, 1992; Huse et al., Science, 246:1275-1281, 1989; Griffiths et al., EMBO J., 12:725-734, 1993.

Additionally, recombinant antibodies, such as chimeric and humanized monoclonal antibodies, including both human and non-human portions, which can be made using standard recombinant DNA techniques, are provided herein. Such chimeric and humanized monoclonal antibodies can be produced by recombinant DNA techniques known in the art, for example using methods described in WO 87/02671; European Patent Application 184,187; European Patent Application 171,496; European Patent Application 173,494; WO 86/01533; U.S. Pat. No. 4,816,567; European Patent Application 125,023; Better et al., Science, 240:1041-1043, 1988; Liu et al., Proc. Natl. Acad. Sci. USA 84:3439-3443, 1987; Liu et al., J. Immunol., 139:3521-3526, 1987; Sun et al., Proc. Natl. Acad. Sci. USA, 84:214-218, 1987; Nishimura et al., Canc. Res., 47:999-1005, 1987; Wood et al., Nature, 314:446-449, 1985; and Shaw et al., J. Natl. Cancer Inst., 80:1553-1559, 1988); Morrison, Science, 229:1202-1207, 1985; Oi et al., Bio/Techniques, 4:214, 1986; U.S. Pat. No. 5,225,539; Jones et al., Nature, 321:552-525, 1986; Verhoeyan et al., Science, 239:1534, 1988; and Beidler et al., J. Immunol., 141:4053-4060, 1988.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a RPTPσ. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG IgA, and IgE antibodies. For an overview of this technology for producing human antibodies, see Lonberg and Huszar (Int. Rev. Immunol., 13:65-93, 1995). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al., Biotechnology, 12:899-903, 1994).

An antibody directed against a RPTPσ can be used to detect the polypeptide (e.g., in a cellular lysate or cell supernatant) to evaluate its abundance and pattern of expression. The antibodies can also be used diagnostically to monitor protein levels in tissue as part of a clinical testing procedure, for example, to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H. Similarly, antibodies to sydecan-4 can be used to monitor syndecan-4 levels in tissue as part of a clinical testing procedure (alone or in combination with antibodies against RPTPσ), e.g., to determine the efficacy of a given treatment regimen.

A test agent/compound that has been screened by a method described herein and determined to modulate RPTPσ expression, clustering or activity, can be considered a candidate compound. A candidate compound that has been screened, e.g., in an in vivo model of a RA, and determined to have a desirable effect on the disorder, can be considered a candidate therapeutic agent. Candidate therapeutic agents, once screened in a clinical setting, are therapeutic agents. Candidate therapeutic agents and therapeutic agents can be optionally optimized and/or derivatized, and formulated with physiologically acceptable excipients to form pharmaceutical compositions.

The compounds described herein that can modulate RPTPσ expression, clustering or activity can be incorporated into pharmaceutical compositions. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminotetraacetic acid; buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active agent/compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation can include vacuum drying or freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Dosage units can also be accompanied by instructions for use.

Toxicity and therapeutic efficacy of such compounds can be determined using known pharmaceutical procedures in cell cultures or experimental animals (animal models of RA, for example). These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in to minimize potential damage to uninfected cells and, thereby, reduce side effects.

The data obtained from the cell culture assays and animal studies can be used in formulating a range of dosage for use in humans. The dosage of such compounds lies generally within a range of circulating concentrations that include the ED50 with little or no toxicity. The dosage may vary within this range depending upon the dosage form employed and the route of administration utilized. For a compound used as described herein (e.g., for treating RA in a subject), the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In a particular embodiment, the disclosure provides a method to treat a disease or disorder that has proteoglycan irregularity associated with receptor protein tyrosine phosphatase sigma (RPTPσ) activation, comprising: contacting a RPTPσ of an abnormal fibroblast cell with an RPTPσ agent that inhibits ectodomain clustering and/or promotes PTP activity of RPTPσ but does not inhibit ectodomain clustering and/or promotes functional activity of RPTPσ from a normal fibroblast cell or OA FLS cell. In an alternate embodiment, the disclosure provides a method to treat a disease or disorder that has proteoglycan irregularity associated with receptor protein tyrosine phosphatase sigma (RPTPσ) inactivation comprising: contacting a RPTPσ of an abnormal fibroblast cell with an RPTPσ agent that promotes ectodomain clustering and/or inhibits PTP activity of the RPTPσ of the abnormal fibroblast cell. In one or more embodiments disclosed herein, the abnormal and normal fibroblast cells are Fibroblast-Like Synoviocyte (FLS) cells. In one or more embodiments disclosed herein, the disorder or disease is selected from rheumatoid arthritis, idiopathic pulmonary fibrosis, Dupuytren's disease, scleroderma or cancer. In one or more embodiments disclosed herein, the disorder or disease is Rheumatoid arthritis.

In a further embodiment, the disclosure provides a method to determine whether a disease or disorder that has proteoglycan abnormality associated with receptor protein tyrosine phosphatase sigma (RPTPσ) can be treated with a RPTPσ compound or RPTPσ agent, comprising: contacting an abnormal fibroblast cell with the RPTPσ compound or RPTPσ agent in vitro; determining whether the RPTPσ compound or RPTPσ agent modulates (increases or decreases) ectodomain clustering and/or inhibit or increases functional activity and/or the PTP activity of RPTPσ. In a further embodiment, the method includes measuring the amount or changes in the amount of syndecan-4 in a biological sample from the subject. In a further embodiment, the disclosure provides a method to determine whether a disease or disorder that has proteoglycan abnormality associated with receptor protein tyrosine phosphatase sigma (RPTPσ) inactivation can be treated with a RPTPσ compound or RPTPσ agent, comprising: contacting a RPTPσ of an abnormal fibroblast cell with the RPTPσ compound or RPTPσ agent in vitro; determining whether the RPTPσ compound or RPTPσ agent inhibit ectodomain oligomerization of the RPTPσ of the abnormal fibroblast cell but does not inhibit ectodomain oligomerization of a RPTPσ from a normal or OA fibroblast cell. In one or more embodiments disclosed herein, the abnormal and normal fibroblast cells are Fibroblast-Like Synoviocyte (FLS) cells. In one or more embodiments disclosed herein, the disorder or disease is selected from rheumatoid arthritis, idiopathic pulmonary fibrosis, Dupuytren's disease, and scleroderma or cancer. In one or more embodiments disclosed herein, the disorder or disease is rheumatoid arthritis.

The disclosure also provides a method of determining a prognosis of a subject undergoing therapy to treat rheumatoid arthritis, idiopathic pulmonary fibrosis, Dupuytren's disease, scleroderma or cancer, the method comprising determining the amount of ectodomain clustering and/or functional activity of the RPTPσ before and after treatment with a drug, therapy or test agent, wherein a decrease in ectodomain clustering and/or increase in functional activity of the RPTPσ is indicative of a beneficial prognosis. In one embodiment, the method comprises obtaining a sample from the subject. In further embodiment, the sample is blood or tissue.

The invention is illustrated in the following examples, which are provided by way of illustration and are not intended to be limiting.

### EXAMPLES

Cell Culture. Fibroblast-like synoviocytes are isolated and cultured from synovium of patients with RA and OA. Briefly, the collected synovial tissues are finely minced into pieces and transferred to a tissue culture flask in Dulbecco's modified Eagle's medium (DMEM) (Hyclone Laboratories, Losan, Utah, USA) supplemented with 10% fetal bovine serum (FBS) (Hyclone Laboratories). Within 14 days, FLSs migrated out from the tissue explant and formed confluent monolayers. At approximately 80% confluency, FLSs are subsequently trypsinized, collected, re-suspended, and plated for expansion. FLSs between the third and fifth passage typically demonstrate morphological characters under phase contrast microscope and the expression level of CD55 should be over 95% using a flow cytometry method.

FLS Transwell Migration Assays. The transwell migration assay is a commonly used test to study the migratory response of endothelial cells to angiogenic inducers or inhibitors. This assay is also known as the Boyden or modified Boyden chamber assay. During this assay, endothelial cells are placed on the upper layer of a cell permeable membrane and a solution containing the test agent is placed below the cell permeable membrane. Following an incubation period (3-18 hours), the cells that have migrated through the membrane are stained and counted. The main advantage of this assay is its detection sensitivity.

Migration ability of FLSs is measured in a transwell cell culture chamber apparatus with 8 μm pore membrane (Costar, New York, N.Y., USA). Briefly, FLSs were seeded at a density of 5×10⁴ cells/mL in six-well plates. Twelve hours later, FLSs are trypsinized, collected, and re-suspended with serum-free medium. The cell suspension (5×10³ cells/mL) is loaded into the upper chamber of the transwell insert. Medium containing 10% FBS (600 μL) is added to the lower compartment as a chemoattractant. After 8 h of incubation, the filters are removed and cells remaining on the upper surface of the membrane are removed with a cotton swab. The cells adhering beneath the membrane are fixed in 4% paraformaldehyde and stained with crystal violet for 30 min. Migration ability of FLSs was quantified by cell counts of five random fields at 100 magnifications in each membrane.

Scratch-wound assay. The scratch-wound assay is a simple, reproducible assay commonly used to measure basic cell migration parameters such as speed, persistence, and polarity. Cells are grown to confluence and a thin “wound” introduced by scratching with a pipette tip. Cells at the wound edge polarize and migrate into the wound space. Advantages of this assay are that it does not require the use of specific chemoattractants or gradient chambers and it generates a strong directional migratory response, even in cell types that do not show robust responses in “single cell” migration assays. It is most reliably analyzed when performed using time-lapse imaging, which can also yield valuable cell morphology/protein localization information.

Flow-cytometry assay. Flow cytometry is a laser based, biophysical technology employed in cell counting, sorting, biomarker detection and protein engineering, by suspending cells in a stream of fluid and passing them by an electronic detection apparatus. When sample solution is injected into a flow cytometer, the particles are randomly distributed. The sample is ordered into a single particle stream then can be interrogated by the machine's detection system. After hydrodynamic focusing, each particle passes through one or more beams of light. Light scattering or fluorescence emission (assumed the particle is labeled by a fluorochrome) provides information about the particle's properties. Fluorescence measurements taken at different wavelengths can provide quantitative and qualitative data about fluorochrome-labeled cell surface receptors or intracellular molecules such as DNA and cytokines. The specificity of detection is controlled by optical filters, which block certain wavelengths while transmitting others. A major application of flow cytometry is to separate cells according to subtype or epitope expression for further biological studies. This process is called cell sorting. In a typical flow cytometry sample preparation, phosphate buffered saline (PBS) is a common suspension buffer and the most straightforward samples for flow cytometry include non-adherent cells from culture, bacteria, yeast, blood and tissues. For cell culture, the growth of cells should better be 10⁵-10⁷ cells/ml to prevent the flow cytometer from clogging up for the sorting speed is about 2,000-20,000 cells/second.

Statistical analysis. Comparisons between stimulated and unstimulated samples are performed using paired Student's t-test and expression in RA vs. OA samples using unpaired Student's t-test. For cell proliferation experiments, Two-way ANOVA is used and for cell cycle analysis, multiple t-tests with Holm-Sidak correction for multiple comparisons is used. Data is analyzed using GraphPad Prism 6.0 and considered statistically significant if P<0.05.

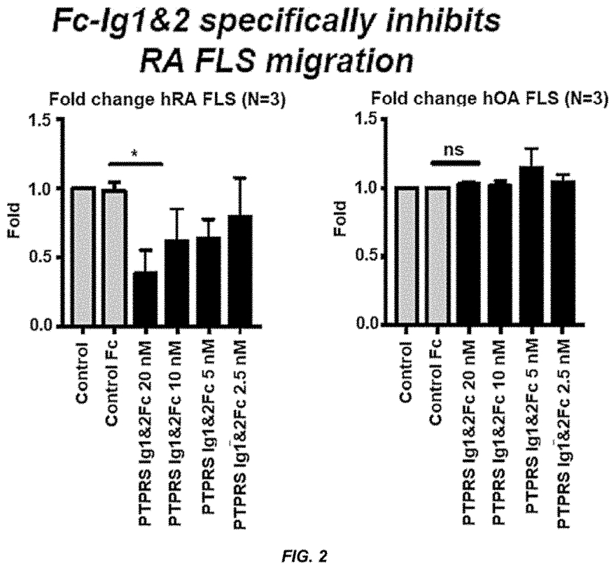

RA FLS, but not OA FLS, show dose dependent inhibition by RPTPσ Ig1&2 in a Transwell Migration Assay. FLS were allowed to migrate through uncoated transwell chambers in response to 5% FBS in the presence of RPTPσ Ig1&2 at various concentrations (2.5 nM, 5 nM, 10 nM, and 20 nM) or vehicle/IgG1Fc as control. For visualization, cells are either pre-stained with 2 μM CellTracker Green™ or stained post-invasion with 2 μM Hoechst for 30 min at room temperature. Fluorescence of migrating cells on each membrane was visualized as above. The results of the transwell migration assay demonstrate that RPTPσ Ig1&2-Fc specifically inhibits RA FLS migration in a dose response manner, while RPTPσ Ig1&2-Fc did not inhibit migration of OA FLS in a dose dependent manner (see FIG. 2).

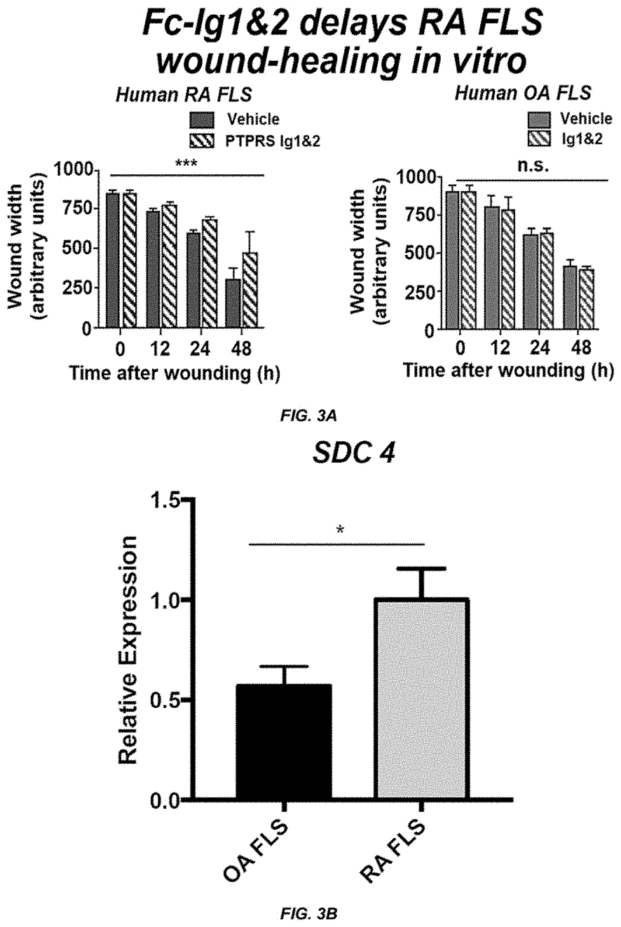

RPTPσ Ig1&2 specifically delays RA FLS healing in comparison to OA FLS in a scratch-wound assay. RA FLS and OA FLS were grown on tissue culture plates using growth media (DMEM with 10% FBS) until they formed confluent monolayers. The growth media was removed and replaced with serum free media. The serum starved cells were scratch-wounded. The serum free media was then replaced with media comprising 10% FBS. The cells were grown in the presence of vehicle or 20 nM RPTPσ IG1&2. The wound width was then measured at three time points (12 h, 24 h, and 48 h). RPTPσ Ig1&2 was shown to specifically delay RA FLS healing in comparison to OA FLS (see FIG. 3).

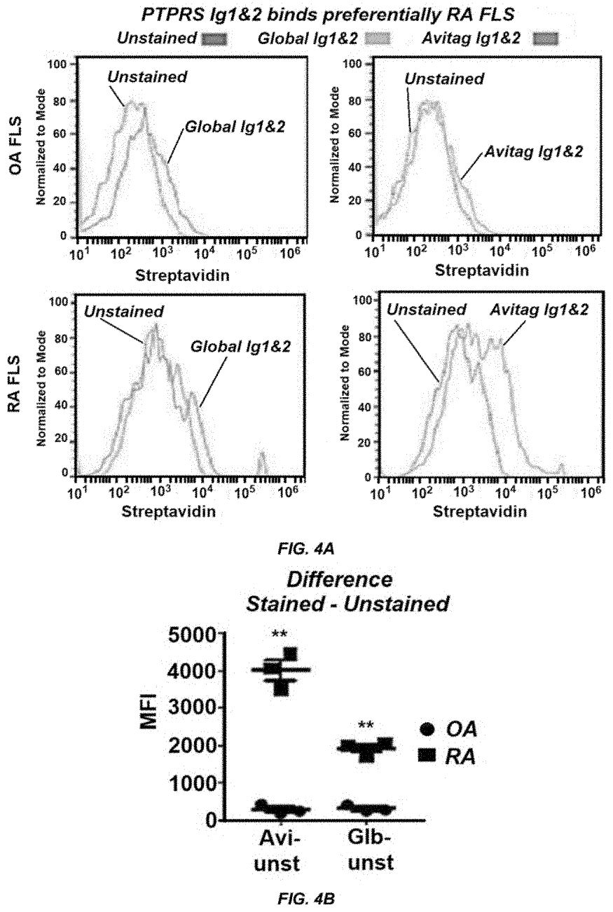

RPTPσ Ig1&2 binds preferentially RA FLS. RA FLS (N=3) and OA FLS (N=3) were stained with chemically Biotinylated Ig1&2 and Avitag Biotinylated Ig1&2. All data are expressed as normalized to mode. Representative flow diagrams of the comparison between staining of Biotinylated RPTPσ Ig1&2 in OA and RA FLS were analyzed by FACS. It was found that RPTPσ Ig1&2 binds preferentially RA FLS (see FIG. 4A-B).

It will be understood that various modifications may be made without departing from the spirit and scope of the invention. Other embodiments are within the scope of the following claims.

1. A method of treating arthritis in a subject, the method comprising:
obtaining a biological sample comprising synovial cells, synovial-like cells and/or synovial fluid from the joint of the subject;
contacting the biological sample with a test agent that inhibits clustering and/or promotes PTP activity of receptor protein tyrosine phosphatase sigma (RPTPσ); and
determining whether (i) there is a change in the clustering and/or biological activity of RPTPσ, or (ii) whether the agent binds to a ligand of the RPTPσ ectodomain;
wherein if there is an inhibition of RPTPσ clustering and/or increase in PTP activity, or binding of the test agent to the ligand of the RPTPσ ectodomain the subject is treated with an agent that inhibits RPTPσ clustering or promotes PTP activity and/or RPTPσ biological activity.

2. The method of claim 1, wherein the agent is a soluble extracellular domain of RPTPσ.

3. The method of claim 1, wherein the agent is an RPTPσ Ig1&2 polypeptide.

4. The method of claim 1, wherein the agent is an antibody that specifically interacts with the RPTPσ ectodomain and inhibits clustering.

5. The method of claim 1, further comprising measuring the level of syndecan-4.

6. A method of screening a subject having or at risk of having arthritis, the method comprising:
obtaining fibroblast-like synoviocytes (FLS) from a subject;
contacting the FLS cells with an agent that inhibits clustering and/or promotes PTP activity of receptor protein tyrosine phosphatase sigma (RPTPσ), and
determining whether (i) there is a change in the clustering and/or biological activity of RPTPσ on the FLS cells, or (ii) whether the agent binds to a ligand of the RPTPσ ectodomain on the FLS cells,
wherein if there is an inhibition of RPTPσ clustering and/or increase in PTP activity, or binding of the test agent to a ligand of the RPTPσ ectodomain is indicative of rheumatoid arthritis.

7. The method of claim 6, wherein the agent is a soluble extracellular domain of RPTPσ.

8. The method of claim 6, wherein the agent is an RPTPσ Ig1&2 polypeptide.

9. The method of claim 6, wherein the agent is an antibody that specifically interacts with the RPTPσ ectodomain and inhibits clustering.

10. The method of claim 6, further comprising measuring the level of syndecan-4.

11. (canceled)

12. A method to determine whether a compound or an agent modulates the clustering and/or functional activity of receptor protein tyrosine phosphatase sigma (RPTPσ), comprising:
contacting a RPTPσ of an abnormal fibroblast cell or suspected abnormal fibroblast cell, and a normal or osteroarthritis (OA) fibroblast cell with the compound or the agent; and
determining whether the compound or the agent modulates the clustering and/or functional activity of the RPTPσ of the abnormal fibroblast or suspected abnormal fibroblast cell but does not modulate the clustering and/or functional activity of a RPTPσ from a normal or OA fibroblast cell.

13. The method of claim 12, wherein the method measures whether the compound or the agent promotes the clustering and/or inhibits PTP functional activity of the RPTPσ of the abnormal or suspected abnormal fibroblast cell.

14. The method of claim 12, wherein the method measures whether the compound or the agent inhibits the clustering and/or promotes PTP functional activity of the RPTPσ of the abnormal or suspected abnormal fibroblast cell.

15. The method of claim 12, wherein the fibroblast cell is a fibroblast-like synoviocyte.

16. The method of claim 12, wherein the abnormal fibroblast or suspected abnormal fibroblast is a fibroblast-like synoviocyte from a subject with rheumatoid arthritis.

17. The method of claim 12, wherein the abnormal fibroblast or suspected abnormal fibroblast is a fibroblast from a subject with idiopathic pulmonary fibrosis, Dupuytren's disease, scleroderma or cancer.

18. A pharmaceutical composition comprising:
a compound or agent determined from the method of claim 12, and
a pharmaceutical carrier.

19. (canceled)

20. A method to treat a disease or disorder in a subject that has proteoglycan irregularities associated with receptor protein tyrosine phosphatase sigma (RPTPσ) clustering and/or function activity, comprising:
administering to the subject the pharmaceutical composition of claim 18.

21. The method of claim 20, wherein the disease or disorder is selected from rheumatoid arthritis, idiopathic pulmonary fibrosis, Dupuytren's disease, scleroderma or cancer.

22. The method of claim 21, wherein the disease or disorder is rheumatoid arthritis.
